(19) United States
(12) Patent Application Publication
(10) Pub. No.: US 2023/0061789 A1
(43) Pub. Date: Mar. 2, 2023

(54) SEARCH INFORMATION PROCESSING DEVICE

(71) Applicant: Canon Medical Systems Corporation, Otawara-shi (JP)

(72) Inventors: Katsuhiko FUJIMOTO, Saitama (JP); Yuichi KAWABATA, Nasushiobara (JP)

(73) Assignee: Canon Medical Systems Corporation, Otawara-shi (JP)

(21) Appl. No.: 17/899,692

(22) Filed: Aug. 31, 2022

(30) Foreign Application Priority Data
Sep. 2, 2021 (JP) 2021-143063

(51) Int. Cl. G16H 50/70; G06F 16/9538; G16H 10/60

(52) U.S. Cl. CPC G16H 50/70; G06F 16/9538; G16H 10/60

(57) ABSTRACT

According to one embodiment, a search information processing device includes processing circuitry. The processing circuitry is configured to set disease information of a patient. The processing circuitry is configured to extract descriptions corresponding to the disease information from each of search results of a search site on the Internet. The processing circuitry is configured to append a reliability degree to each of the search results based on the extracted descriptions and evidence information for the disease information.

### CROSS-REFERENCE TO RELATED APPLICATION

This application is based upon and claims the benefit of priority from the prior Japanese Patent Application No. 2021-143063, filed Sep. 2, 2021, the entire contents of which are incorporated herein by reference.

### FIELD

Embodiments described herein relate generally to a search information processing device.

### BACKGROUND

In general, when a patient is notified of a disease by a doctor, particularly in the case of a serious disease, the patient often searches for information on the Internet because the patient is not able to understand and is unconvinced only by a doctor's explanation. However, especially in the case of serious diseases such as cancers and diseases such as the recent novel coronavirus infection, various information from correct evidence-based information to false information like fraudulent information flows on the Internet. Therefore, when a patient searches for information on the Internet, the patient acquires a search result in which the various information pieces described above are listed.

However, in such a search result, information at the higher level of the search is not necessarily correct and reliable, and information at the lower level of the search is not necessarily incorrect and unreliable. In general, patients cannot determine authenticity or reliability of specialized information on diseases, and it is difficult for the patients to appropriately select search results.

### BRIEF DESCRIPTION OF THE DRAWINGS

FIG. 1 is a block diagram showing a search information processing device according to a first embodiment and a peripheral configuration thereof.

FIG. 2 is a block diagram showing a configuration of the search information processing device according to the first embodiment.

FIG. 3 is a schematic diagram showing an example of an explanatory screen of evidence information according to the first embodiment.

FIG. 4 is a block diagram showing a configuration of a management device according to the first embodiment.

FIG. 5 is a schematic diagram showing an electronic medical record storage unit in the first embodiment.

FIG. 6 is a schematic diagram showing a medical DB storage unit in the first embodiment.

FIG. 7 is a schematic diagram showing an ID storage unit in the first embodiment.

FIG. 8 is a sequence diagram showing an example of an operation in the first embodiment.

FIG. 9 is a flowchart showing an example of an operation of step ST13 in the first embodiment.

FIG. 10 is a flowchart showing an example of an operation of step ST17 in the first embodiment.

FIG. 11 is a schematic diagram showing a display example of a screen in the first embodiment.

FIG. 12 is a schematic diagram showing an example of a screen to be superimposed and displayed on the screen shown in FIG. 11.

FIG. 13 is a schematic diagram showing an example of a screen to be superimposed and displayed on the screen shown in FIG. 12.

FIG. 14 is a flowchart showing an example of an operation of step ST13 in a second embodiment.

FIG. 15 is a flowchart showing an example of an operation of step ST13 in a third embodiment.

FIG. 16 is a schematic diagram showing an example of a screen in a third embodiment.

FIG. 17 is a flowchart showing an example of an operation of step ST13 in a fourth embodiment.

FIG. 18 is a schematic diagram showing an example of a screen in a fourth embodiment.

### DETAILED DESCRIPTION

In general, according to one embodiment, a search information processing device includes processing circuitry. The processing circuitry is configured to set disease information of a patient. The processing circuitry is configured to extract descriptions corresponding to the disease information from each of search results of a search site on the Internet. The processing circuitry is configured to append a reliability degree to each of the search results based on the extracted descriptions and evidence information for the disease information.

Embodiments will be described below with reference to the drawings.

### First Embodiment

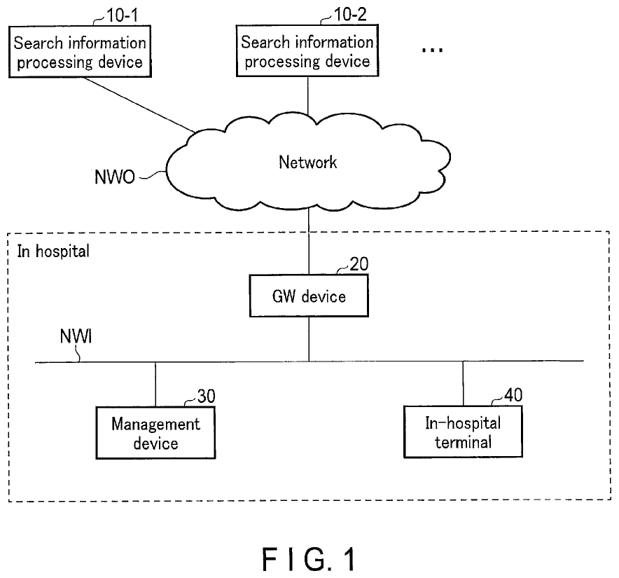

FIG. 1 is a block diagram showing a search information processing device according to a first embodiment and a peripheral configuration thereof. In FIG. 1, a plurality of search information processing devices 10-1, 10-2, etc. are connected to a management device 30 and an in-hospital terminal 40 via a network NWO such as the Internet out of the hospital, an in-hospital gateway (GW) device 20, and a network NWI. Each of the search information processing devices 10-1, 10-2, etc. is a terminal device such as a personal computer or a smartphone used by a patient or patient's family. These search information processing devices have similar configurations, and thus the search information processing device 10 from which a branch number is omitted will be described as a representative example.

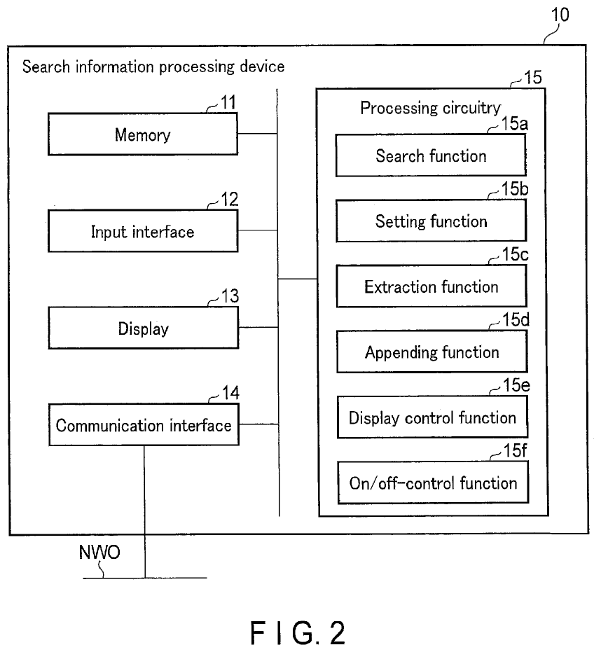

As shown in FIG. 2, the search information processing device 10 comprises a memory 11, an input interface 12, a display 13, a communication interface 14, and processing circuitry 15.

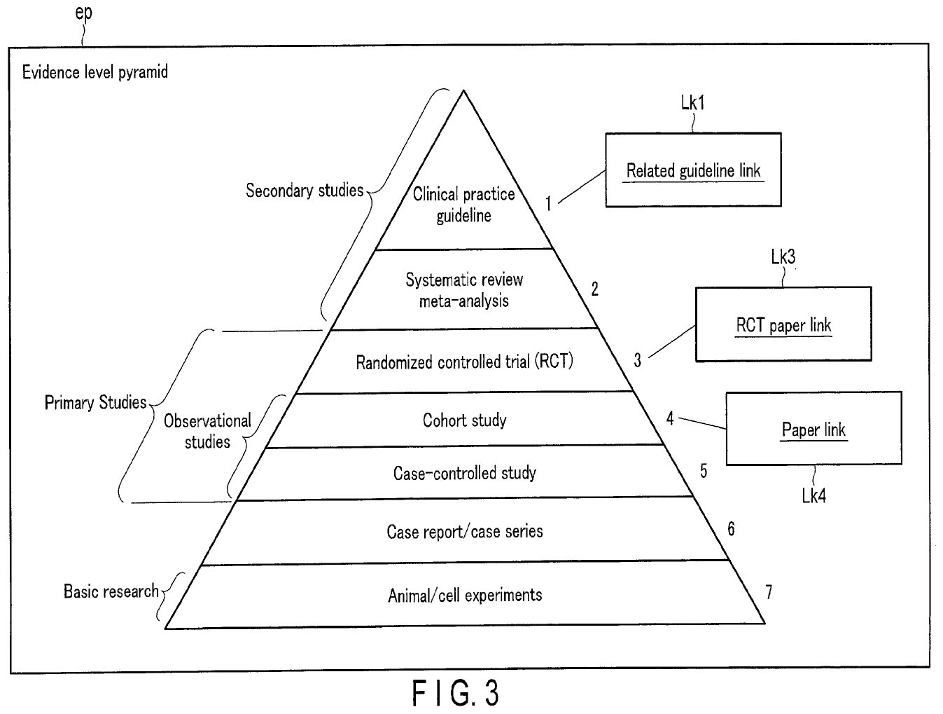

The memory 11 comprises memory bodies for recording electrical information such as ROM (Read Only Memory), RAM (Random Access Memory), HDD (Hardware Disk Drive), and an image memory, and peripheral circuits such as a memory controller and a memory interface attached to the memory bodies. The memory 11 stores various programs such as a program of the search information processing device 10, a browser that mutually communicates with a website such as a search site, and a mail application that receives notifications such as e-mails from other devices, and various data such as data before processing, data in the middle of processing, data after processing, data to be stored in advance, and tables. For example, as shown in FIG. 3, screen data for displaying an explanatory screen ep for evidence information, and the like can be used as appropriate as the data to be stored in advance.

The explanatory screen ep is displayed based on screen data in which screen data for explaining an evidence level pyramid representing evidence levels 1 to 7 obtained by ranking medical bases is provided with links Lk1, Lk3 and Lk4 to guideline/papers in association with the levels. Specifically, the evidence level is an indicator that ranks the strength of conclusions by classification of studies. The evidence level “1” represents the strongest conclusion (medical basis), and as the ranking of the evidence level decreases, the evidence level represents a weaker conclusion (medical basis). In FIG. 3, the evidence levels 1 and 2 correspond to secondary studies. Specifically, the evidence level 1 corresponds to clinical practice guidelines, and the evidence level 2 corresponds to a systematic review meta-analysis. The evidence levels 3 to 5 correspond to primary studies, and the evidence levels 4 and 5 out of the evidence levels 3 to 5 correspond to observational studies. Specifically, the evidence level 3 corresponds to a randomized controlled trial (ROT). The evidence level 4 corresponds to a cohort study, and the evidence level 5 corresponds to a case-control study. The evidence level 6 corresponds to case report/case series. The evidence level 7 corresponds to a basic research, specifically corresponds to animal/cell experiments. In the evidence level pyramid, studies with higher evidence levels are supported by the accumulation of studies with lower evidence levels.

Returning to FIG. 2, the input interface 12 is implemented by a track ball, a switch button, a mouse, a keyboard, a touch pad (or track pad) that performs an input operation by touching an operation surface, a touch panel display (or touch screen) in which a display screen and a touch pad are integrated with each other, or the like, which is configured to input various instructions, commands, information, selections, and settings from a user (a patient or Patient's family) into the search information processing device 10. The input interface 12 is connected to the processing circuitry 15, converts an input operation received from the user into an electric signal, and outputs the electric signal to the processing circuitry 15. In this case, the input interface 12 may display, on the display 13, a user interface (GUI: Graphical User Interface) which is used for the user to input various instructions by a physical operation component such as a mouse or a keyboard. In the present specification, the input interface 12 is not limited to a member having a physical operating component. For example, examples of the input interface 12 include an electric signal processing circuitry that receives an electric signal corresponding to an input operation from an external input device provided separately from the search information processing device and outputs the electric signal to the processing circuitry 15. In the following description, “an operation of the input interface 12 by a user” is also referred to as “user's operation”.

The display 13 comprises a display main body that displays a search screen, a search result, an explanatory screen ep, and the like, an internal circuit that supplies a display signal to the display main body, and peripheral circuits such as a connector and a cable that connect the display main body and the internal circuit. The display 13 is controlled to display by the processing circuitry 15, and can display any data as appropriate. The display 13 is an example of a display unit.

The communication interface 14 is a circuit for connecting the search information processing device 10 to a network NWO such as the Internet to communicate with a search site such as Google (registered trademark) or another device. In the following description, the description that the communication interface 14 intervenes in the communication between the search information processing device 10 and other devices will be omitted.

The processing circuitry 15 reads out programs in the memory 11 based on an instruction input via the input interface 12 by a user, and controls the search information processing device 10 according to these programs. For example, the processing circuitry 15 is a processor that implements respective functions of the search information processing device 10 according to the programs read out from the memory 11. The respective functions include, for example, a search function 15a, a setting function 15b, an extraction function 15c, an appending function 15d, a display control function 15e, an on/off control function 15f, and the like. For example, the processing circuitry 15 may further execute a browser for causing the computer to implement the search function 15a. Further, the processing circuitry 15 may execute programs for causing the computer to implement the setting function 15b for setting patient's disease information, the extraction function 15c for extracting descriptions corresponding to the disease information from respective search results of a search site on the Internet, and the appending function for appending reliability degrees to the respective search results based on the extracted descriptions and evidence information for the disease information. The respective functions may be implemented while distributed to a plurality of processors as appropriate. Alternatively, the respective functions or some of the respective functions may be executed as appropriate by another device. For example, the search function 15a and the on/off control function 15f out of the respective functions may be executed by another device.

Next, the search function 15a, the setting function 15b, the extraction function 15c, the appending function 15d, the display control function 15e, and the on/off control function 15f as the respective functions will be described in order. However, share of the respective functions described below is for convenience's sake, and can be changed as appropriate. This is because even if processing to be shared by a function is shared by another function, there is no change in that the processing circuitry 15 still executes the processing. For example, the search function 15a, the setting function 15b, the extraction function 15c, and the appending function 15d may display their data in the middle of processing or after processing, and the like on the display 13 by executing the function of the display control function 15e as appropriate. Further, the extraction function 15c may be switched to an on-state or an off-state according to a user's operation by executing the function of the on/off control function 15f as appropriate. The fact that the share of the respective functions can be changed is the same in the following embodiments and modifications.

The search function 15a accesses a search site on the Internet according to a user's operation, inputs a search condition such as a keyword on the displayed search screen, and sends a confirmed search conditions to the search site. Thereafter, a search result returned from the search site is displayed on the display 13 by the display control function 15e. However, when the setting function 15b and the extraction function 15c described later are in an ON-state, the search function 15a stores the search result returned from the search site in the memory 11 without displaying the search result.

The setting function 15b sets disease information of a patient according to a user's operation. Here, the setting function 15b may set the disease information based on an electronic medical record of the patient. Further, the setting function 15b may transmit a patient ID indicating the patient or a family ID indicating the family of the patient, and a terminal ID indicating a terminal of the patient or the family to medical facilities that the patient has visited or has been hospitalized in, receive disease information as a response to the transmission, and set the received disease information. The setting function 15b and the processing circuitry 15 are an example of a setting unit.

The extraction function 15c extracts descriptions corresponding to the disease information from the respective search results of a search site on the Internet. For example, the extraction function 15c may extract descriptions matching with or similar to the description included in the disease information out of the descriptions included in the search results. The extraction function 15c and the processing circuitry 15 are an example of an extraction unit.

The appending function 15d appends a reliability degree to each of the search results based on the extracted descriptions and the evidence information for the disease information. Here, the appending function 15d may append a validity level of each extracted description to the extracted description based on the evidence information. The “reliability degree” described here is an index indicating the level of a medical basis for each search result. The “validity level” is an index indicating the level of a medical basis of each description contained in each search result. Giving some supplemental description, the reliability degree of the search result is an index based on the validity level of each description. For example, when the search result contains one description and the validity level of one description is “1” whereas the validity level of the other description is “3”, the reliability degree is “1” or “3” according to which description is a main explanation. However, when a plurality of descriptions contradict one another, the reliability degree is set to “none”. Each of the reliability degree and the validity level may be, for example, an evidence level in the clinical research. Further, the reliability degree may also be referred to as certification information. The appending function 15d and the processing circuitry 15 are an example of an appending unit.

The display control function 15e causes the display 13 to display various data. For example, the display control function 15e sorts the respective search results to which the reliability degrees are appended according to the reliability degrees, and causes the display 13 to display each of the sorted search results. Here, the display control function 15e may control the display 13 so that search results having appended reliability degrees equal to or less than a threshold value among the sorted search results are not displayed. Further, when any of the sorted search results is selected, the display control function 15e causes the display 13 to display descriptions contained in the selected search result, and causes the display 13 to display validity levels appended to the extracted descriptions out of the above descriptions. Further, the display control function 15e may cause the display 13 to display at least one of an explanation of the evidence information and a link destination of the evidence information. The display control function 15e and the processing circuitry 15 are an example of a display control unit. Out of configurations containing the respective functions as described above, particularly configurations including the extraction function 15c and the appending function 15d may be referred to as “reliability degree appending filter” or “evidence level filter”. This configuration supports the selection of each search result by processing of appending a reliability degree, a validity level, an explanation, link, and performing sorting of search order, filtering, etc. without changing the description content of the search results of the search site. Since reliability degree/validity level/explanation/link is appended to the search result in a state where a character string included in the search result is maintained, the description content of the search result is not changed. Since the sort of the search order and the filtering of the search result are executed for each search result as a unit, the description content of the search result is not changed.

The on/off control function 15f performs on/off control on the extraction function 15c according to a user's operation. The on/off control function 15f and the processing circuitry 15 are an example of a control unit.

Further, the gateway device 20 is a device that communicably connects the network NWI in the hospital and the network NWO out of the hospital.

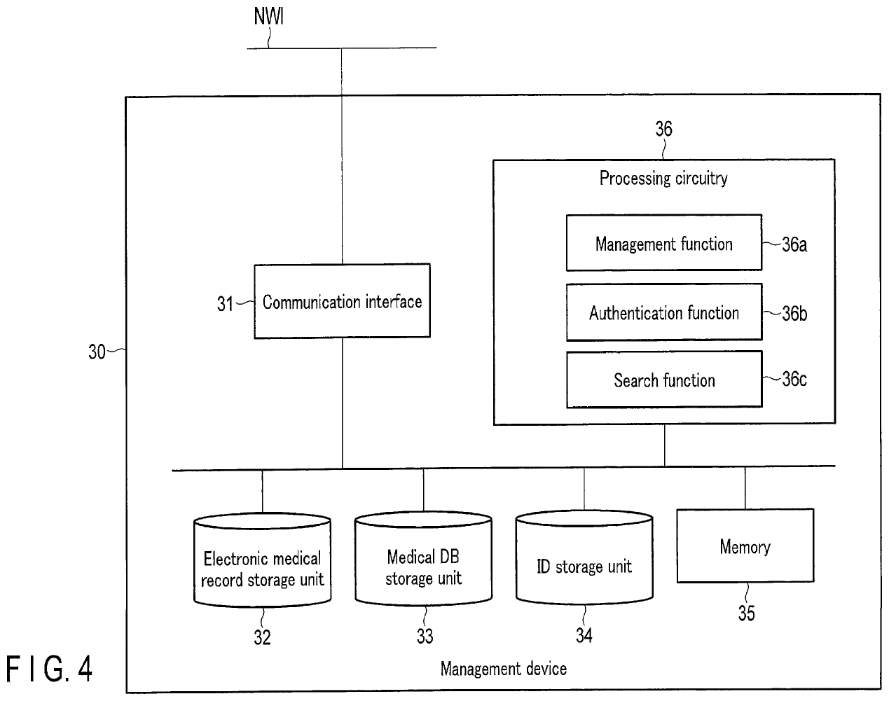

As shown in FIG. 4, the management device 30 comprises a communication interface 31, an electronic medical record storage unit 32, a medical DB storage unit 33, an ID storage unit 34, a memory 35, and processing circuitry 36.

The communication interface 31 is a circuit for connecting the management device 30 to the network NWI to communicate with other devices. For example, a network interface card (NIC) is usable as the communication interface 31. In the following description, the description that the communication interface 31 intervenes in the communication between the management device 30 and other devices will be omitted.

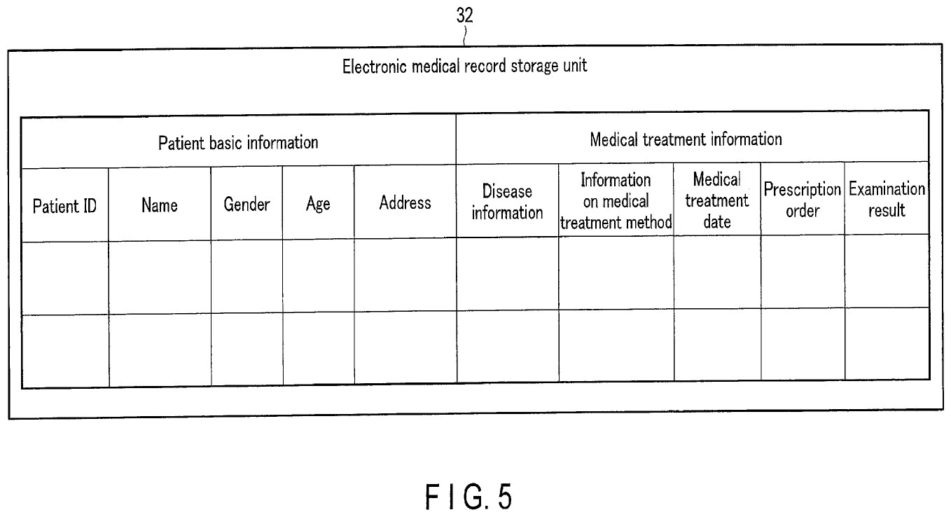

As shown in FIG. 5, the electronic medical record storage unit 32 is a storage device or a storage region therein, which stores an electronic medical record for each patient. In the following description, electronic medical records of a plurality of patients are stored in the electronic medical record storage unit 32. In FIG. 5, each row of an electronic medical record corresponds to an electronic medical record for each patient.

Here, the electronic medical record includes, for example, patient basic information and medical treatment information. The patient basic information includes patient ID, name, gender, age and address for each patient. Note that ID is an abbreviation for identification information. For example, a patient number including a numeral or an alphanumeric character can be used as the patient ID. However, the patient ID is not limited to the patient number, and a combination of information pieces that can uniquely identify a patient, such as a combination of name, date of birth, and gender, may be used, or a social security and tax number may be used.

The medical treatment information includes, for example, disease information (disease name, symptom) of a patient, information on a medical treatment method for the disease, and the like, and may include necessary items such as medical treatment date, prescription order, examination result, medical history (not shown), medical treatment expenses, preferences, allergies, and a family history as appropriate.

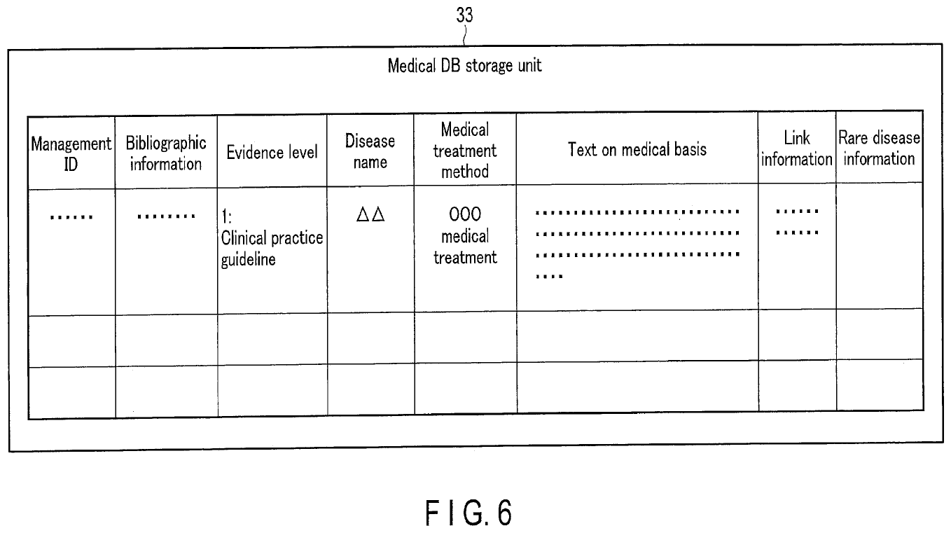

As shown in FIG. 6, the medical DB storage unit 33 is a storage device or a storage region therein, which stores medical information for each research paper as a medical DB. In the following description, medical information of a plurality of research papers is stored as a medical DB. In FIG. 6, each row of the medical DB corresponds to each research paper.

Here, the medical DB includes, for example, management ID, bibliographic information, evidence level, disease name, medical treatment method, text on medical basis (a main part of research paper), link information, and rare disease information for each research paper. For example, a management number including a numeral or an alphanumeric character is usable as the management ID. However, a number which does not overlap with the patient ID is preferable as the management ID. The bibliographic information is information that identifies a research paper, and includes, for example, author name, paper name, publication name, issuance country, issuance place, issuance date, volume number, issue number, page and the like. However, the bibliographic information is not limited to these information, and for example, in the case of research papers published electronically, the bibliographic information may include author name, title, related part (page, column, line, item number, figure number, or first and last words and phrases), publication date (issuance date), publisher (issuer), publication place (issuance place), link destination of the research paper (full text), and the like. The evidence level is set along the definition of an evidence level pyramid according to which research stage the evidence of a research paper corresponds to. Information on the disease name and the medical treatment method is set according to the content of the research paper. The text on the medical basis is set by extracting the main part of the research paper. The link information is information indicating the link destination of the research paper (full text). The rare disease information is set when the disease is a rare disease, and includes, for example, information indicating a hospital or doctor who frequently diagnoses the disease. The rare disease information is not limited to this information, and the rare disease information may include the number of clinical cases reported for the particular disease. In place of the medical DB, the medical DB storage unit 33 may store electronic data of each research paper.

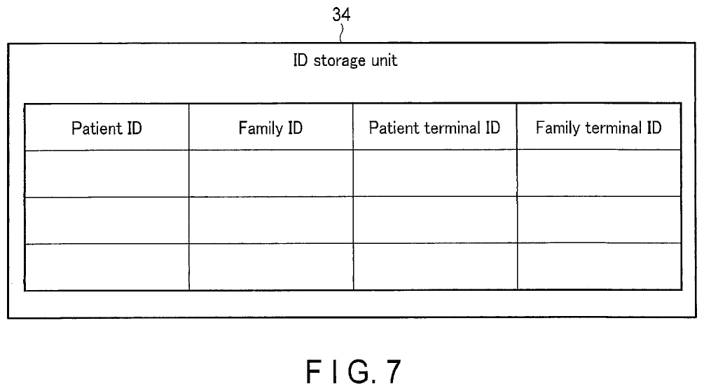

As shown in FIG. 7, the ID storage unit 34 is a storage device or a storage region therein, which stores patient ID, family ID, patient terminal ID, and family terminal ID in association with one another for each patient in an ID table. In FIG. 7, each row of the ID table corresponds to each patient. Further, FIG. 7 shows a case where there is only one family member other than the patient. However, the present invention is not limited to this case, and even when there are a plurality of family members other than the patient, it can be dealt with by increasing the number of columns in the ID table.

The patient ID is identical to the patient ID described in the electronic medical record.

The family ID is information that can identify the patient's family, and for example, a family number including a numeral or an alphanumeric character can be used.

The patient terminal ID is information that can identify the search information processing device 10 to be operated by the patient. For example, when the search information processing device 10 is a smartphone, a terminal code (IMEI) or the like can be used, and when the search information processing device 10 is a personal computer, a MAC address or the like can be used as appropriate. IMEI is an abbreviation for international mobile equipment identifier. MAC is an abbreviation for media access control.

The family terminal ID is information that can identify the search information processing device 10 to be operated by the patient's family, and for example, the terminal code (IMEI), MAC address or the like of the search information processing device 10 can be used as appropriate.

The memory 35 comprises a memory body for recording electrical information such as a ROM (Read Only Memory), a RAM (Random Access Memory), an HDD (Hardware Disk Drive), and an image memory, and peripheral circuits such as a memory controller and a memory interface which are appended to the memory body. The memory 35 stores various programs such as a program of the management device 30, and various data such as data before processing, data in the middle of processing, data after processing, data to be stored in advance, and tables. Not limited to a case where the memory 35 is a member separated from the electronic medical record storage unit 32, the medical DB storage unit 33 and the ID storage unit 34 described above, and the memory 35 may include the electronic medical record storage unit 32, the medical DB storage unit 33 and the ID storage unit 34.

The processing circuitry 36 reads out a program in the memory 35 and controls the management device 30 according to the program. For example, the processing circuitry 36 is a processor that implements each function of the management device 30 according to a program read out from the memory 35. The functions include, for example, a management function 36a for the electronic medical record storage unit 32, the medical DB storage unit 33 and the ID storage unit 34, an authentication function 36b based on the ID storage unit 34, a search function 36c for the electronic medical record storage unit 32 and the medical DB storage unit 33.

The management function 36a newly registers or updates the electronic medical record in the electronic medical record storage unit 32 for each patient according to an operation of the in-hospital terminal 40. Further, the management function 36a newly registers or updates the medical DB in the medical DB storage unit 33 for each research paper according to an operation of the in-hospital terminal 40. Further, the management function 36a newly registers or updates the ID table in the ID storage unit 34 for each patient according to an operation of the in-hospital terminal 40. The management function 36a and the processing circuitry 36 are an example of a management unit.

The authentication function 36b refers to the ID storage unit 34 based on the patient ID or family ID received from the search information processing device 10 and the terminal ID, and authenticates the search information processing device 10 as a valid device when the patient ID or family ID and the terminal ID are located on the same row in the ID table. The authentication function 36b and the processing circuitry 36 are an example of an authentication unit.

When the authentication result by the authentication function 36b is valid, the search function 36c searches the electronic medical record storage unit 32 based on the patient ID in the ID table which corresponds to the patient ID or family ID received from the search information processing device 10, and transmits a search result to the search information processing device 10.

Further, the search function 36c searches the medical DB storage unit 33 based on disease information received from the search information processing device 10, and transmits a search result to the search information processing device 10. The search function 36c and the processing circuitry 36 are an example of a search unit.

The in-hospital terminal 40 is a computer terminal or workstation connected to the network NWI in the hospital, and comprises a communication interface, a memory, an input interface, a display, and processing circuitry (not shown). The in-hospital terminal 40 accesses the management device 30 according to an administrator's operation, whereby information is newly registered or updated in the electronic medical record storage unit 32, the medical DB storage unit 33, and the ID storage unit 34 via the processing circuitry 36 of the management device 30.

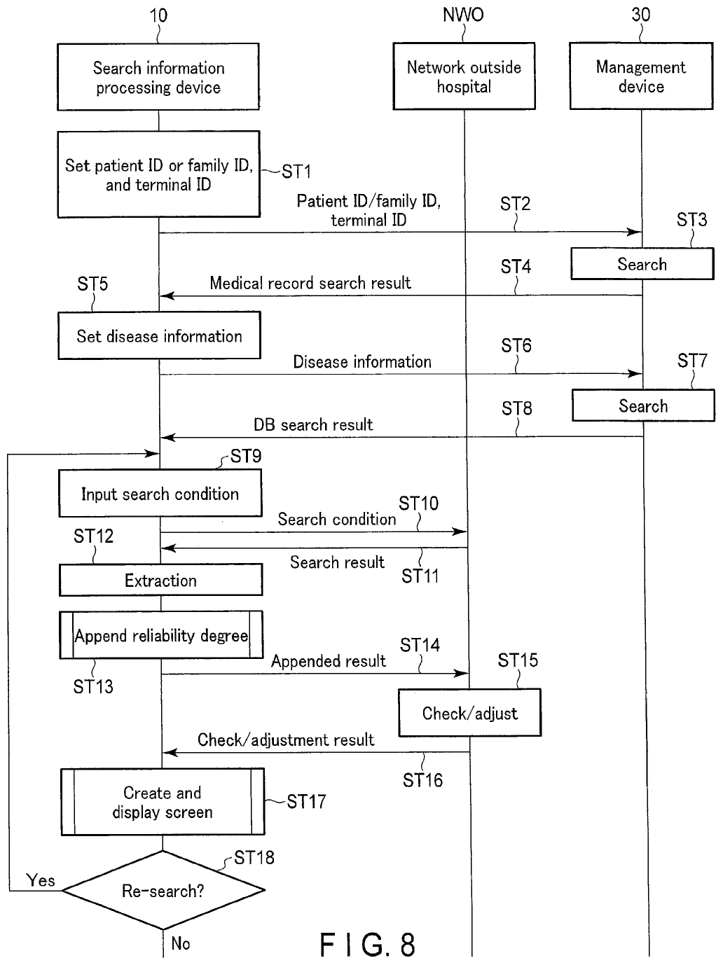

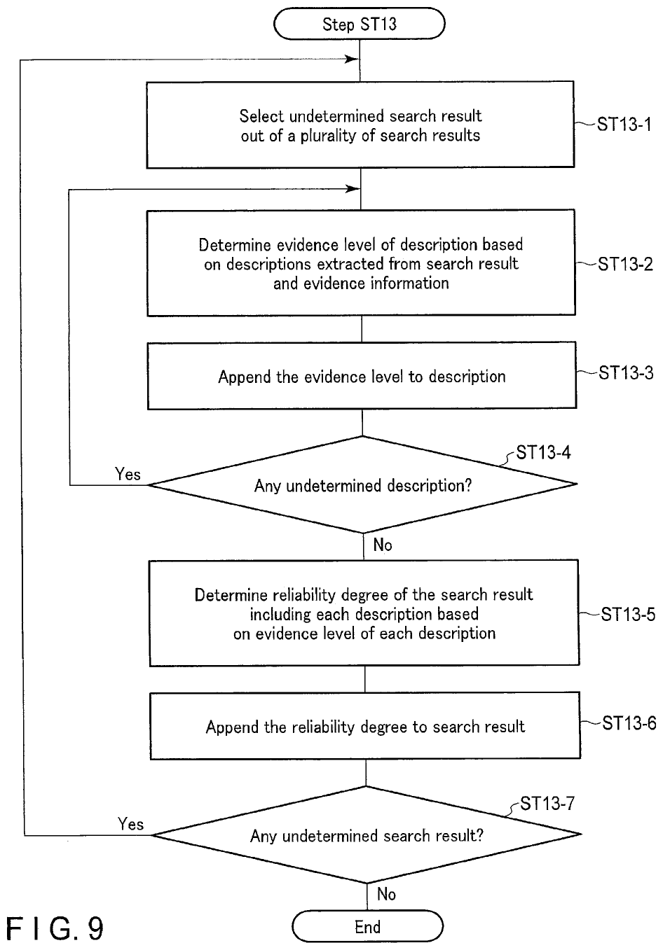

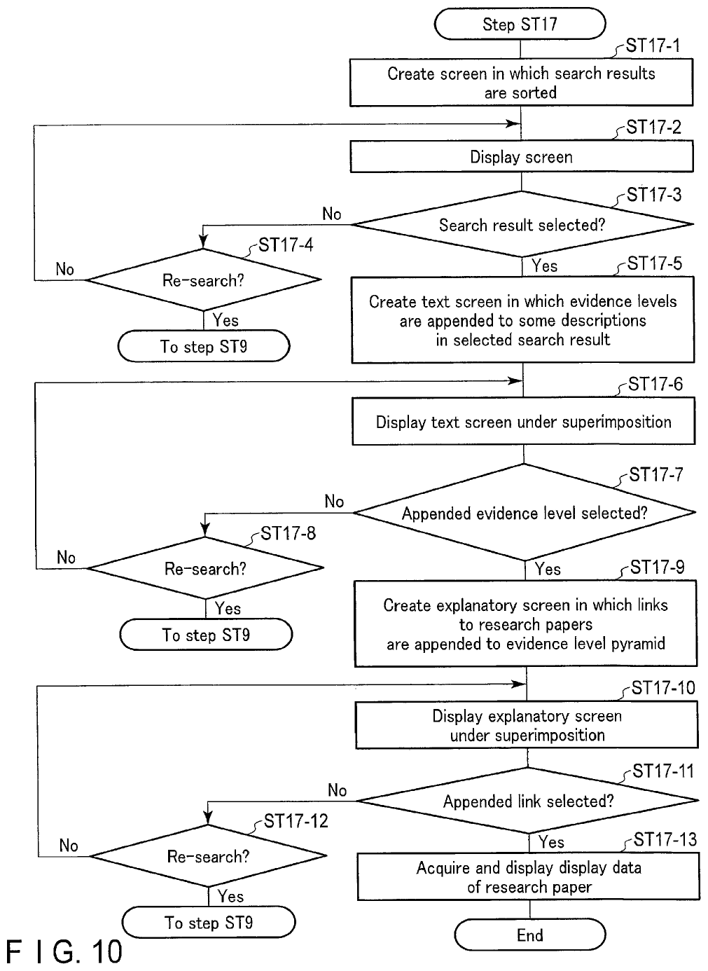

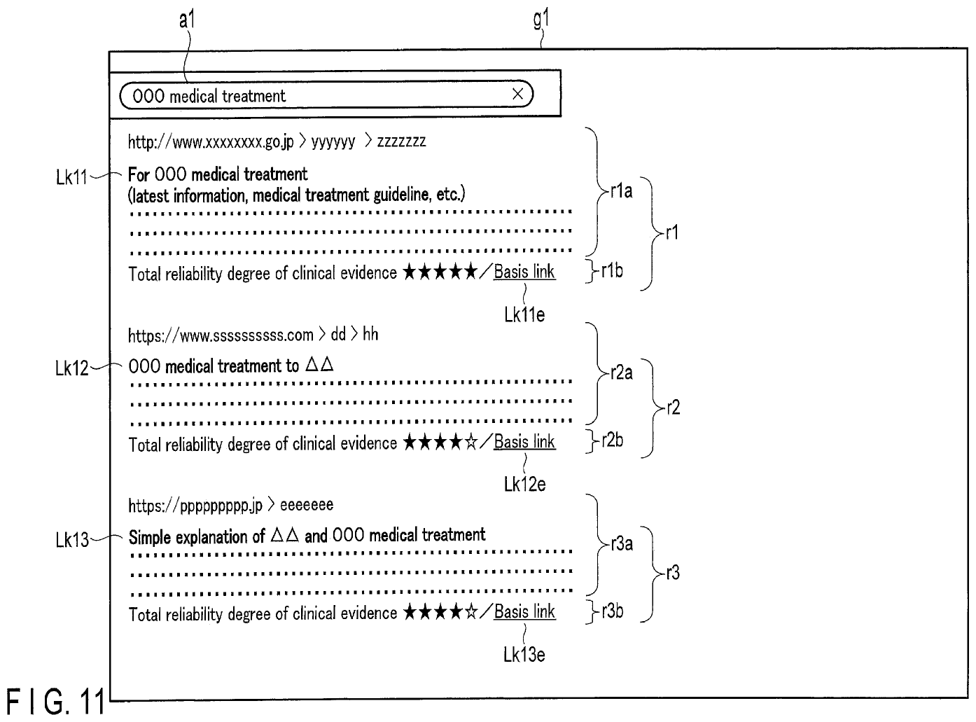

Next, the operation of the search information processing device configured as described above and the peripheral configuration thereof will be described with reference to a sequence diagram of FIG. 8, flowcharts of FIGS. 9 and 10, and schematic diagrams of FIGS. 11 to 13.

First, after a patient has a medical examination at a hospital, the patient receives an explanation (notification) about his/her disease and an explanation about a medical treatment method from a doctor. The doctor writes disease information of the patient into an electronic medical record and stores the electronic medical record in the electronic medical record storage unit 32 by operating the in-hospital terminal 40. Further, in the management device 30, it is assumed that a medical database for research papers is stored in the medical database storage unit 33 and an ID table for patients is stored in the ID storage unit 34.

Further, a patient has a time to undergo a medical treatment while visiting the hospital and a time to wait for a medical treatment after returning home, except for cases such as immediate hospitalization and surgery. Here, it is assumed that the patient searches for information on his/her disease on the Internet by using his/her search information processing device 10 such as a personal computer at home or a smartphone. At this time, the search information processing device 10 sets the extraction function 15c of the processing circuitry 15 to ON-state by the patient's operation on the input interface 12. Under this state, step ST1 is started.

In step ST1, the processing circuitry 15 of the search information processing device 10 sets a patient ID and a patient terminal ID by the patient's operation on the input interface 12. The processing circuitry 15 may set a patient's family ID and a family terminal ID according to an operation by a patient's family member instead of the patient.

After the step ST1, in step ST2, the search information processing device 10 transmits the patient ID and the patient terminal ID to the hospital that the patient has visited or has been hospitalized in. Note that when the family ID and the like are set in step ST1, the search information processing device 10 transmits the family ID and the family terminal ID to the hospital that the patient has visited or has been hospitalized in.

After the step ST2, in step ST3, the processing circuitry 36 of the management device 30 refers to the ID storage unit 34 based on the transmitted patient ID and patient terminal ID, and authenticates the search information processing device 10 as a valid device when the patient ID and the patient terminal ID are located on the same row of the ID table. Likewise, when receiving the family ID and the family terminal ID, the processing circuitry 36 refers to the ID storage unit 34 based on the family ID and the family terminal ID to authenticate whether the search information processing device 10 is a valid device or not. In any case, if the authentication result indicates invalidity, the management device 30 terminates the processing. On the other hand, when the authentication result indicates validity, the processing circuitry 36 searches the electronic medical record storage unit 32 based on the patient ID in the ID table which corresponds to the patient ID or the family ID received from the search information processing device 10, and acquires a search result.

After the step ST3, in step ST4, the management device 30 transmits the search result of the electronic medical record to the search information processing device 10. The search result of the electronic medical record includes disease information of the patient. The search result of the electronic medical record is not limited to the disease information of the patient, but may include an underlying disease (background disease) of the patient.

After the step ST4, in step ST5, the processing circuitry 15 of the search information processing device 10 receives the search result of the electronic medical record, and sets the disease information of the patient based on the search result. The disease information includes, for example, information on a disease name and a medical treatment method. Note that the disease information to be set may include disease information of the underlying disease (background disease) in addition to the direct disease information. In this case, it is possible to further consider the influence on the medical treatment method presented by the doctor based on the patient's own background disease. When setting the background disease of the patient, it may be extracted and set from the search result of the electronic medical record, or may be set by the patient's operation on the input interface 12.

After the step ST5, in step ST6, the search information processing device 10 transmits the set disease information to the management device 30.

After the step ST6, in step ST7, the processing circuitry 36 of the management device 30 searches the medical DB storage unit 33 based on the transmitted disease information, and obtains a search result.

After the step ST7, in step ST8, the management device 30 transmits the search result of the medical DB to the search information processing device 10. The search information processing device 10 receives the search result of the medical DB, and stores the search result in the memory 11. The search result of the medical DB includes evidence information for disease information. The evidence information includes, for example, an evidence level, information on a medical treatment method, and information on a medical basis. The evidence information may further include bibliographic information and link information of research papers. In addition, the evidence information may further include rare disease information.

After the step ST8, in step ST9, the processing circuitry 15 of the search information processing device 10 accesses a search site on the Internet which is a network NWO outside the hospital, by the operation on the input interface 12 by the patient (or family member), and inputs a search condition such as keywords on a search screen displayed on the display 13. The search condition includes keywords related to disease information such as a disease name and information on a medical treatment method.

After the step ST9, in step ST10, the search information processing device 10 transmits a settled search condition to the search site by operating the input interface 12.

After the step ST10, in step ST11, the processing circuitry 15 of the search information processing device 10 receives each of search results from the search site and stores each of the search results in the memory 11. Each of the search results is a medical article about disease information. However, each of the search results by the search site includes various information such as medically/academically reliable information, articles and blogs written based on biased impressions by other ordinary people, and information at various times from latest information to old information.

After the step ST11, in step ST12, the processing circuitry 15 extracts descriptions corresponding to the disease information set in step ST5 from the respective search results of the search site which are stored in the memory 11. For example, the extraction function 15c extracts descriptions that match with or are similar to the description included in the disease information of the electronic medical record among the descriptions included in the respective search results of the search site.

After the step ST12, in step ST13, the processing circuitry 15 appends a reliability degree to each of the search results based on the extracted descriptions and the evidence information for the disease information. Here, the processing circuitry 15 may append a validity level for each extracted description to the extracted description based on the evidence information. In the following description, each of the reliability degree and the validity level is assumed to be an evidence level in the clinical research. Such step ST13 is executed, for example, by steps ST13-1 to ST13-7 as shown in FIG. 9.

In other words, the processing circuitry 15 selects a search result the reliability degree of which has not been determined, among a plurality of search results by the search site (step ST13-1).

After the step ST13-1, based on the description extracted from the selected search result and the evidence information, the processing circuitry 15 determines the evidence level of the description (step ST13-2). For example, if the extracted description has a content corresponding to evidence information of the evidence level “1”, the evidence level thereof is determined to be “1”. Further, if the extracted description has a content corresponding to evidence information of the evidence level “3”, the evidence level thereof is determined to be “3”. Further, if there is no evidence information corresponding to the extracted description like a case where a completely false description is extracted, the evidence level is determined to be “none”.

After the step ST13-2, the processing circuitry 15 appends the determined evidence level to the description (step ST13-3).

After the step ST13-3, the processing circuitry 15 determines whether a description the evidence level of which has not been determined is present in the selected search result (step ST13-4). If there is a description the evidence level of which has not been determined, the processing circuitry 15 returns to ST13-2. On the other hand, if the result of the determination in step ST13-4 is negative, the processing circuitry 15 proceeds to step ST13-5.

After the step ST13-4, based on the evidence levels appended to the respective descriptions included in the selected search result, the processing circuitry 15 determines the reliability degree of the search result including the respective descriptions (step ST13-5). For example, when the evidence level appended to each of a plurality of descriptions included in the search result is “1” or “3”, the reliability degree of the search result is determined as “1” or “3” according to which one of the evidence levels “1” and “3” descriptions serving as a major explanation has. For example, if the evidence levels of nine descriptions are “1” and the evidence level of one description is “3”, the reliability degree is determined as “1”. Further, for example, when the evidence level appended to each of a plurality of descriptions included in the search result varies between “1” and “7” and also includes a plurality of “none”, the reliability degree is determined as “none”. If one description shows a positive view and the evidence level thereof is “6”, whereas the other description shows a negative view and the evidence level thereof is “6”, the reliability degree is determined as “6”. In addition to this, each research paper which is a basis for the evidence level of each of different opinions may be specified, and the specified result may be displayed on the display 13 in step ST17 or the like described later.

After the step ST13-5, the processing circuitry 15 appends the determined reliability degree to the description (step ST13-6).

After the step ST13-6, the processing circuitry 15 determines whether a search result the reliability degree of which has not been determined is present in the search results by the search site (step ST13-7). If there is a search result the reliability degree of which has not been determined, the processing circuitry 15 returns to step ST13-1. On the other hand, if the result of the determination in step ST13-7 is negative, step ST13 including steps ST13-1 to ST13-7 is terminated.

Returning to FIG. 8, after the step ST13, in step ST14, the search information processing device 10 transmits a result appended in step ST13 is transmitted to, for example, a telemedicine system (not shown). Note that the appended result is each search result including each description to which the evidence level is appended, and having the reliability degree appended thereto. Further, the telemedicine system includes, for example, an operation terminal to be operated by, for example, a doctor or a specialized expert (hereinafter, also referred to as a doctor or the like).

After the step ST14, in step ST15, the operation terminal of the telemedicine system receives an appended result and displays it on the display, and checks/adjusts the appended result according to the operation of a doctor or the like.

After the step ST15, in step ST16, the operation terminal of the telemedicine system returns the checked/adjusted result to the search information processing device 10. The checked/adjusted result is each search result including each description to which the evidence level is appended, and having the reliability degree appended thereto, and the appended evidence level and reliability degree have been checked/adjusted by the doctor or the like.

After the step ST16, in step ST17, the processing circuitry 15 of the search information processing device 10 sorts the respective search results to which reliability degrees have been appended, according to the reliability degrees, creates a screen including each of the sorted search results, and causes the display 13 to display the screen. Here, the processing circuitry 15 may control the display 13 so as not to display search results having appended reliability degrees equal to or less than a threshold value among the sorted search results. Further, when any of the sorted search results is selected, the processing circuitry 15 may cause the display 13 to display descriptions included in the selected search result, and also cause the display 13 to display the evidence level appended to the extracted description out of the descriptions. Further, the processing circuitry 15 may cause the display 13 to display at least one of an explanation of the evidence information and a link destination of the evidence information. Such step ST17 is executed, for example, by steps ST17-1 to ST17-12 as shown in FIG. 10.

In other words, as shown in FIG. 11, the processing circuitry 15 sorts, according to the reliability degrees r1b to r3b, the r1 to r3 of the search results r1a to r3c to which the reliability degrees r1b to r3b have been appended, and creates a screen g1 including the r1 to r3 of the sorted search results (step ST17-1). In the example of FIG. 11, the reliability degree r1b represents the evidence level “1” with five stars. The reliability degrees r2b and r3b represent the evidence level “2” with four stars. Although not shown, the reliability degree represents, for example, the evidence level “3” with three starts, the evidence level “4” with two starts, the evidence level “5” with one and a half stars, the evidence level “6” with one start, and the evidence level “7” with a half start. However, the correspondence relation between the number of stars representing the reliability degree and the evidence level may be changed as appropriate.

Thereafter, the processing circuitry 15 causes the display 13 to display the screen g1 (step ST17-2). In FIG. 11, the screen g1 includes an input area a1 for inputting a search condition such as a keyword. The search results r1a to r3a by the search site include titles Lk11 to Lk13 for selecting the search results (and developing articles). Further, the appended reliability degrees r1b to r1b may include links Lk11e to Lk13e for developing bases of the reliability degrees.

The processing circuitry 15 determines whether any of (the titles Lk11 to Lk13 of) the search results within the screen g1 is selected (step ST17-3). If the determination is negative and re-searching is performed, the processing circuitry 15 returns to step ST9 (step ST17-4: Yes). If re-searching is not performed (step ST17-4: No), the screen display in step ST17-2 is continued. On the other hand, for example, when the title Lk11 is selected in step ST17-3, the processing circuitry 15 proceeds to step ST17-5.

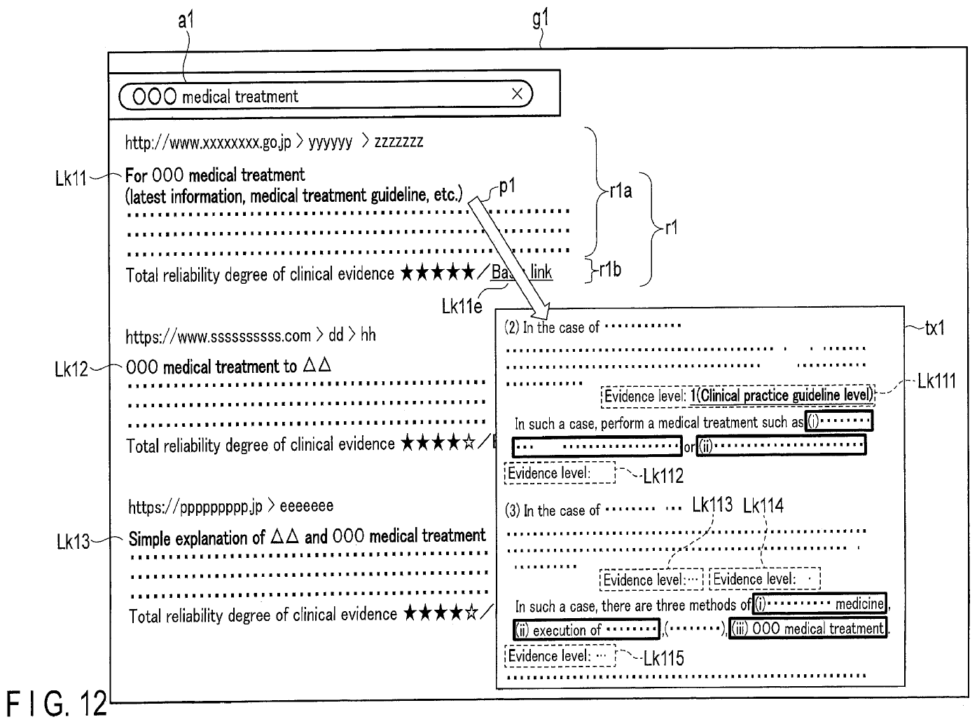

As indicated by an arrow p1 in FIG. 12, the processing circuitry 15 creates a text screen tx1 in which evidence levels Lk111 to Lk115 are appended to a part of the description of the search result r1a for which the title Lk11 is selected (step ST17-5). In FIG. 12, the evidence levels Lk111 to Lk115 appended to the description include a link for selecting the evidence level and expanding an explanatory screen.

Thereafter, the processing circuitry 15 controls the display 13 so that the text screen tx1 is displayed while superimposed on a part of the screen g1 (step ST17-6).

The processing circuitry 15 determines whether any of the evidence levels Lk111 to Lk115 in the text screen tx1 is selected (step ST17-7). If the determination is negative and re-searching is performed, the processing circuitry returns to step ST9 (step ST17-8: Yes). If re-searching is not performed (step ST17-8: No), the processing circuitry 15 continues the superimposed display of step ST17-6. On the other hand, if any of the evidence levels Lk111 to Lk115 is selected in step ST17-7, the processing circuitry 15 proceeds to step ST17-9. Here, for example, it is assumed that the evidence level Lk111 is selected.

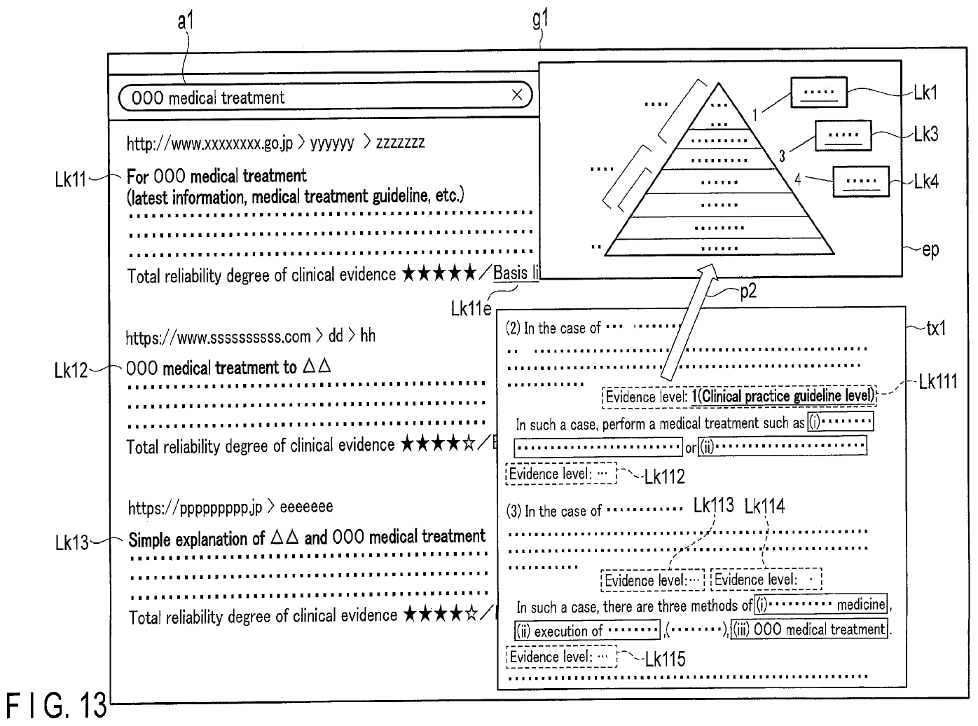

As indicated by an arrow p2 in FIG. 13, the processing circuitry 15 creates an explanatory screen ep in which links Lk1, Lk3, and Lk4 to research papers are appended to an evidence level pyramid (step ST17-9). The links Lk1, Lk3, and Lk4 to the research papers are included in the search results (evidence information) of the medical DB received in step ST8.

Thereafter, the processing circuitry 15 controls the display 13 so as to display the explanatory screen ep1 while the explanatory screen ep1 is superimposed another part of the screen g1 (step ST17-10).

The processing circuitry 15 determines whether any of the links Lk1, Lk3, and Lk4 in the explanatory screen ep is selected (step ST17-11). If the determination is negative and re-searching is performed, the processing circuitry 15 returns to step ST9 (step ST17-12: Yes). If re-searching is not performed (step ST17-12: No), the processing circuitry 15 continues the superimposed display in step ST17-10. On the other hand, if any of the links Lk1, Lk3, and Lk4 is selected in step ST17-11, the processing circuitry 15 proceeds to step ST17-13. Here, for example, it is assumed that the evidence level Lk1 is selected.

The processing circuitry 15 acquires display data of a research paper based on the selected link, and causes the display 13 to displays the display data (step ST17-13). As a result, the step ST17 including steps ST17-1 to ST17-13 is terminated.

Returning to FIG. 8, after the step ST17, in step ST18, the processing circuitry 15 of the search information processing device 10 returns to step ST9 if re-searching is performed (step ST18: Yes). If re-searching is not performed, the processing circuitry 15 continues the screen display in step ST17.

As described above, according to the first embodiment, the disease information of the patient is set, and the descriptions corresponding to the disease information are extracted from the respective search results of the search site on the Internet. Further, the reliability degrees are appended to the respective search results based on the extracted descriptions and the evidence information for the disease information.

In this way, with the configuration of appending the reliability degree to each of the search results of the search site based on the evidence information for the disease information, it is possible to support appropriate selection of the search results when searching for the disease.

Giving some supplemental description, in the first embodiment, not a doctor or a specialized expert, but a person with little medical expertise such as a patient or patient's family, friend or acquaintance is assumed as a main user. Here, for example, in the case of a patient, even when the patient searches for specialized information about his/her disease by using a conventional user terminal, it is difficult for the patient to judge the authenticity of the acquired search result, and the patient is easily tossed about by a lot of information included in various search results. For this reason, it is difficult for the patient to reach reliable information, and the patient may be caught in suspicious information under unknown authenticity. On the other hand, according to the first embodiment, the patient can understand the reliability degree of a search result by the search site with a basis, so that it is possible to greatly reduce risks that the patient is confused by uncertain information, falls into anxiety due to misunderstanding, or is deceived. In addition, the patient deeply and correctly understands medical treatment methods for his/her own diseases, and will be able to undergo medical treatments with greater peace of mind.

Further, according to the first embodiment, the respective search results to which the reliability degrees have been appended are sorted according to the reliability degrees thereof, whereby each of the sorted search results may be displayed on the display. In this case, in addition to the above-mentioned action and effect, highly reliable search results can be displayed at the top of the search. In other words, it is possible to preferentially present highly reliable information based on evidence information to the patient.

Further, according to the first embodiment, the display may be controlled so as not to display search results having appended reliability degrees equal to or less than a threshold value among the sorted search results. In this case, in addition to the above-mentioned action and effect, by performing filtering based on the reliability degree on various search results, it is possible to protect the user from the risk that search results with low reliability degrees are browsed. Further, since search results with low reliability degrees are not displayed, it is possible to substantially restrict access to the search results with low reliability degrees.

Further, according to the first embodiment, a validity level of each extracted description may be appended to the extracted description based on the evidence information. Further, when any of the sorted search results is selected, the display may be caused to display the descriptions included in the selected search result, and also caused to display the validity levels appended to the extracted descriptions out of the descriptions included in the selected search result. In this case, in addition to the above-mentioned action and effect, the description extracted from the search result and the validity level of the description can be displayed.

Further, according to the first embodiment, the display may be caused to display at least one of the explanation of the evidence information and the link destination of the evidence information. In this case, in addition to the above-mentioned action and effect, detailed information regarding the evidence information can be displayed.

According to the first embodiment, each of the reliability degree and the validity level may be the evidence level in a clinical research. In this case, in addition to the above-mentioned action and effect, it can be easily implemented, and also it is possible to enhance the reliability for the reliability degree and the validity level to be appended as compared with a case where special indexes other than the evidence level are appended as the reliability degree and the validity level.

Further, according to the first embodiment, the extraction unit may be subjected to on/off-control according to the user's operation. In this case, it is possible to implement on/off-control according to a search target, for example, like the extraction unit is controlled to be set to ON-state when searching the disease of the patient, and controlled to set to OFF-state when searching diseases of other people which are known from news or the like. Further, when the diseases of other people are searched, appending of the reliability degree and the check/adjustment by a doctor or the like are not executed, so that waste of medical resources can be avoided.

Further, according to the first embodiment, the disease information may be set based on the electronic medical record of the patient. In this case, in addition to the above-mentioned action and effect, it is possible to append more appropriate reliability degree because an appropriate description based on the electronic medical record can be extracted.

Further, according to the first embodiment, the patient ID indicating the patient or the family ID indicating the patient's family, and the terminal ID indicating the terminal of the patient or the patient's family member may be transmitted to medical facilities that the patient has visited or has been hospitalized in, disease information may be received as a response to the transmission, and the received disease information may be set. In this case, in addition to the above-mentioned action and effect, it is possible to support the appropriate selection of the search result by the search site for the patient or the patient's family by the configuration in which the disease information of the patient can be set by only the patient or the patient's family. In other words, the search information processing device can be expanded in application so that the patient's family is not confused by erroneous information or low-evidence information when the patient's family investigates a disease which the patient has contracted. Further, the search information processing device can set disease information of other people regardless of the disease of the patient, and appends reliability degrees to search results. As a result, with respect to evidence levels related to general medical treatment methods for diseases which blood-unrelated persons such as friends contracted, it is possible to append the evidence levels to search results.

The first embodiment may be carried out like the following modification.

A modification of the first embodiment is configured so that the reliability degree is appended to the display result on the display 13 instead of the search result on the Internet like a case that text information such as an electronic book, a news content such as news, or information diagnosed/announced by a doctor is displayed.

In connection with this, the extraction function 15c of the processing circuitry 15 extracts a description corresponding to the disease information from the display result on the display 13. For example, the extraction function 15c may extract descriptions that match or are similar to descriptions included in the disease information out of the descriptions included in the display result.

The appending function 15d appends the reliability degree to the display result based on the extracted descriptions and the evidence information for the disease information. Here, the appending function 15d may append the validity level of each extracted description to the extracted description based on the evidence information. In this case, the display control function 15e may cause the display 13 to display the validity levels appended to the extracted descriptions out of the descriptions included in the display result. Further, the display control function lye may cause the display 13 to display at least one of the explanation of the evidence information and the link destination of the evidence information.

The other configurations are the same as in the first embodiment.

According to the modification of the first embodiment as described above, the same effect as the first embodiment can be acquired for the display result on the display 13 like text information such as an electronic book, a news content such as news, and information diagnosed/announced by a doctor. Note that the modification of the first embodiment may be applied to each of the following embodiments.

### Second Embodiment

Next, a search information processing device according to a second embodiment will be described. Substantially the same parts as those in the above-mentioned figures are designated by the same reference numerals, detailed description thereof will be omitted, and different parts will be described here. Likewise, duplicative description will also be omitted for each of the following embodiments.

The second embodiment is a modification of the first embodiment, and is configured so that when the appended reliability degree is low, the reliability degree is adjusted and appended.

In connection with this, an appending function 15d of processing circuitry 15 performs the following operation in addition to the above-mentioned function. When the disease information indicates a specific disease and the reliability degree before it is appended to the search result is equal to or less than a threshold value, the appending function 15d adjusts the reliability degree before it is appended, based on the number of related presented papers for the specific disease, the rank of a published journal of evidence information for the disease information, and the number of clinical cases reported for the specific disease, and appends the adjusted reliability degree to the search result. Here, for example, an impact factor of an index for measuring the degree of influence of the published journal or a value based on the impact factor can be used as appropriate as the rank of the published journal. Examples of highly-ranked journals include “New England Journal of Medicine,” “The Lancet,” “JAMA (Journal of the American Medical Association), “BMJ (British Medical Journal)”, and “Annals of Internal Medicine”, “JAMA Internal Medicine”, etc.

The other configurations are the same as in the first embodiment.

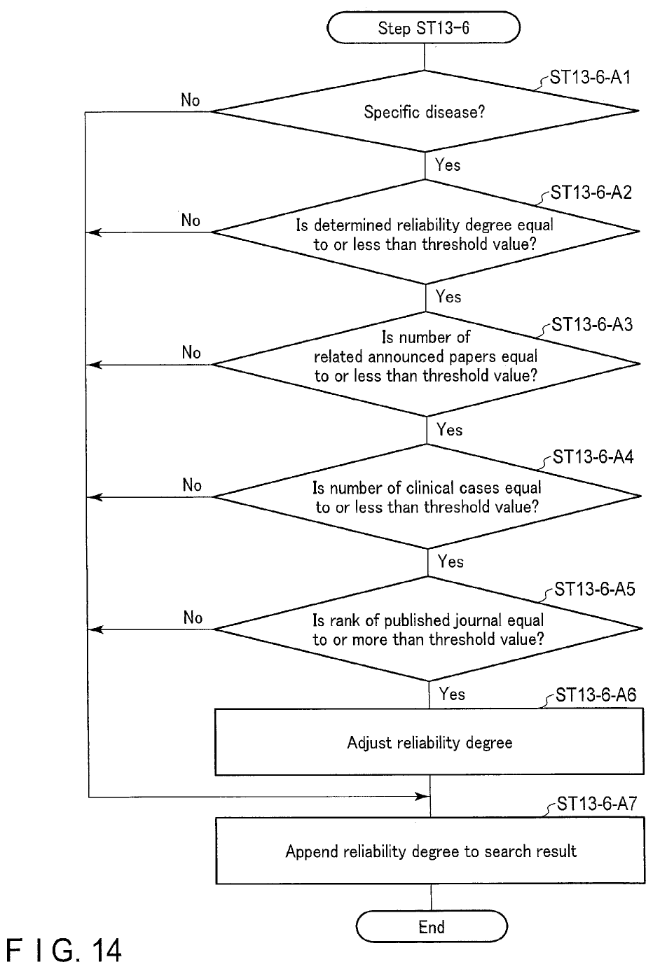

Next, the operation of the search information processing device configured as described above will be described with reference to the flowchart of FIG. 14.

Now, it is assumed that steps ST1 to ST12 are executed in the same manner as described above, and descriptions corresponding to the disease information set in step ST5 are extracted from each of the search results of the search site.

After the step ST12, the step ST13 for appending a reliability degree is executed as shown in FIG. 9. Steps ST13-1 to ST13-5 and ST13-7 of the step ST13 are executed in the same manner as described above. Further, the step ST13-6 is executed, for example, by steps ST13-6-A1 to ST13-6-A7 as shown in FIG. 14.

In other words, the processing circuitry 15 determines whether the disease information set in step ST5 indicates a specific disease (step ST13-6-A1). If the determination is negative, the processing circuitry 15 proceeds to step ST13-6-A7, and if the disease information indicates a specific disease, the processing circuitry 15 proceeds to the next step ST13-6-A2.

The processing circuitry 15 determines whether the reliability degree determined in step ST13-5 is equal to or less than a threshold value (step ST13-6-A2). If the determination is negative, the processing circuitry 15 proceeds to step ST13-6-A7, and if the reliability degree is equal to or less than the threshold value, the processing circuitry 15 proceeds to the next step ST13-6-A3.

The processing circuitry 15 determines whether the number of related presented papers for the specific disease is equal to or less than a threshold value (step ST13-6-A3). If the determination is negative, the processing circuitry 15 proceeds to step ST13-6-A7, and if the number of related presented papers is equal to or less than the threshold value, the processing circuitry 15 proceeds to the next step ST13-6-A4.

The processing circuitry 15 determines whether the number of clinical cases reported for the specific disease is equal to or less than a threshold value (step ST13-6-A4). If the determination is negative, the processing circuitry 15 proceeds to step ST13-6-A7, and if the number of clinical cases is equal to or less than the threshold value, the processing circuitry 15 proceeds to the next step ST13-6-A5.

The processing circuitry 15 determines whether the rank of a published journal of evidence information for the disease information is equal to or more than a threshold value (step ST13-6-A5). If the determination is negative, the processing circuitry 15 proceeds to step ST13-6-A7, and if the rank of the published academic journal is equal to or more than the threshold value, the processing circuitry 15 proceeds to the next step ST13-6-A6.

When the determined reliability degree is low for the specific disease, the processing circuitry 15 makes an adjustment so as to increase a reliability degree before appending in spite of a small number of related published papers or a small number of clinical cases when the rank of the published academic journal is high (step ST13-6-A6).

Thereafter, the processing circuitry 15 appends the adjusted reliability degree to the search result (step ST13-6-A7), and then step ST13 ends.

Hereinafter, steps ST14 to ST18 are executed in the same manner as described above.

As described above, according to the second embodiment, when the disease information indicates a specific disease and the reliability degree before it is appended to the search result is equal to or less than a threshold value, the reliability degree before it is appended is adjusted based on the number of related published papers for the specific disease, the rank of a published journal of evidence information for the disease information, and the number of clinical cases reported for the specific disease, and the adjusted reliability degree is appended to the search result.

In this way, even when the reliability degree of the search result for a specific disease is low, in addition to the effect of the first embodiment, it is possible to append a more appropriate reliability degree by the configuration of adjusting the reliability degree based on the number of related published papers, a published journal, and the number of clinical cases.

### Third Embodiment

Next, a search information processing device according to a third embodiment will be described.

The third embodiment is a modification of the first embodiment, and is configured to display information indicating both a positive paper and a negative paper with respect to a search result when an appended reliability degree is low.

In connection with this, an appending function 15d and a display control function 15e of processing circuitry 15 operate as follows in addition to the above-mentioned functions. When disease information indicates a specific disease and the reliability degree of a search result is equal to or less than a threshold value, the appending function 15d specifies both of a positive paper for the search result and a negative paper for the search result among related published papers for the specific disease.

Further, the display control function 15e causes a display 13 to display information indicating both the specified papers.

The other configurations are the same as in the first embodiment.

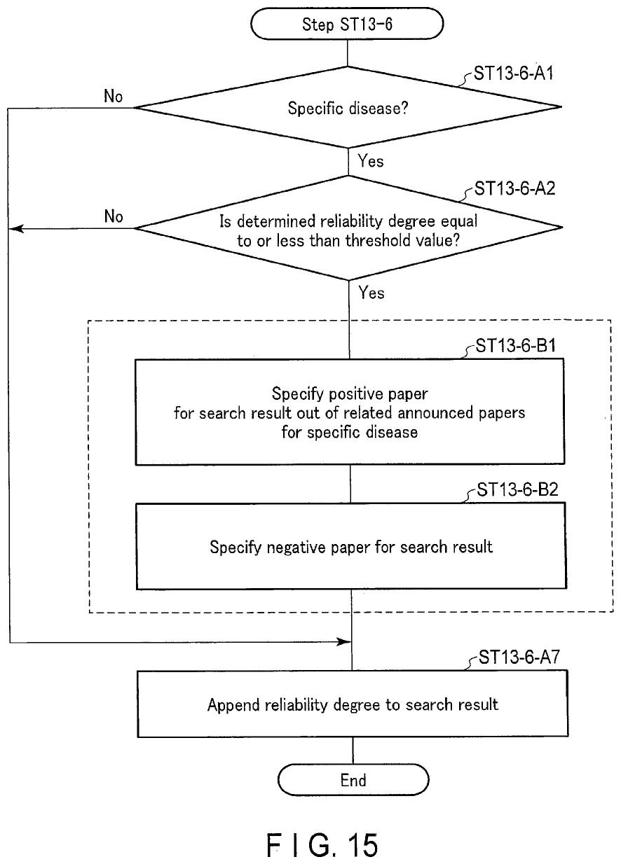

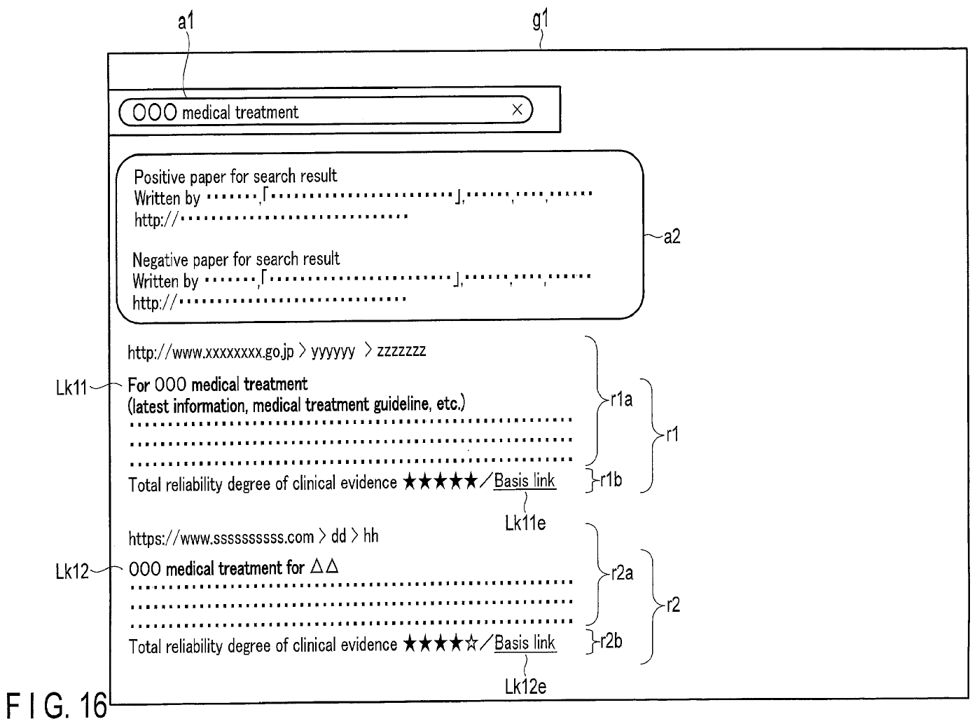

Next, an operation of the search information processing device configured as described above will be described with reference to a flowchart of FIG. 15 and a schematic diagram of FIG. 16.

Now, it is assumed that steps ST1 to ST12 are executed in the same manner as described above, and descriptions corresponding to disease information set in step ST5 are extracted from respective search results of a search site.

After the step ST12, step ST13 for appending a reliability degree is executed as shown in FIG. 9. Steps ST13-1 to ST13-5 and ST13-7 of step ST13 are executed in the same manner as described above. On the other hand, step ST13-6 is executed, for example, by steps ST13-6-A1 to ST13-6-A2, ST13-6-B1 to ST13-6-B2, and ST13-6-A7 as shown in FIG. 15. In FIG. 15, a portion surrounded by a broken line is a portion that is significantly different from FIG. 14.

The processing circuitry 15 determines whether the disease information set in step ST5 indicates a specific disease (step ST13-6-A1). If the determination is negative, the processing circuitry 15 proceeds to step ST13-6-A7, and if the disease information indicates a specific disease, the processing circuitry 15 proceeds to the next step ST13-6-A2.

The processing circuitry 15 determines whether the reliability degree determined in step ST13-5 is equal to or less than a threshold value (step ST13-6-A2). If the determination is negative, the processing circuitry proceeds to step ST13-6-A7, and if the reliability degree is equal to or less than the threshold value, the processing circuitry 15 proceeds to the next step ST13-6-B1.

The processing circuitry 15 specifies a positive paper for the search result among related published papers for the specific disease (step ST13-6-B1). For example, the processing circuitry 15 may specify, as a positive paper, a paper including a text having a high degree of similarity to the text of the search result among the related published papers for the specific disease.

Further, the processing circuitry 15 specifies negative papers for the search result among the related published papers for the specific disease (step ST13-6-B2). For example, the processing circuitry 15 may specify, as a negative paper, a paper other than the positive paper specified in step ST13-6-B1 among the related published papers for the specific disease. Alternatively, a set of papers having opposite views to each other may be described in the medical DB in advance, and according to identification of a paper having a certain view, a paper having an opposite view to the above view may be specified. Further, in steps ST13-6-B1 and ST13-6-B2, the execution order may be reversed.

Thereafter, the processing circuitry 15 appends a reliability degree to the search result (step ST13-6-A7). As a result, the step ST13 ends.

Hereinafter, steps ST14 to ST18 are executed in the same manner as described above. However, in step ST17, the processing circuitry 15 causes the display 13 to display information indicating both the specified positive paper and the specified negative paper. For example, as shown in FIG. 16, the bibliographic information of the positive paper and the bibliographic information of the negative paper may be displayed in an intermediate display area a2 between the input area a1 for the search condition and the search result r1 at the top of the search. Further, for example, the bibliographic information and a link of the positive paper and the bibliographic information and a link of the negative paper may be displayed in the intermediate display area a2.

As described above, according to the third embodiment, when the disease information indicates a specific disease and the reliability degree of the search result is equal to or less than a threshold value, both of a positive paper for the search result and a negative paper for the search result among related published papers for the specific disease are specified. Further, the display is caused to display information indicating both of the specified papers.

In this way, even when the reliability degree of the search result for the specific disease is low, in addition to the effect of the first embodiment, conflicting papers can be compared and described together by the configuration of displaying a positive paper and a negative paper for a search result.

### Fourth Embodiment

Next, a search information processing device according to a fourth embodiment will be described.

The fourth embodiment is a modification of the first embodiment, and it is configured so as to display information on a hospital or doctor who has made a number of medical examinations of a specific disease when the reliability degrees of search results for the specific disease are low or the number of clinical cases of the specific disease is small.

In connection with this, an appending function 15d and a display control function 15e of processing circuitry 15 operate as follows in addition to the above-mentioned functions. When disease information indicates a specific disease and the reliability degree of a search result is equal to or less than a threshold value or the number of clinical cases reported for the specific disease is equal to or less than a threshold value, the appending function 15d specifies information indicating a hospital or doctor who has made a relatively large number of medical examinations of the specific disease. The information indicating the hospital or doctor who has made a relatively large number of medical examinations of the specific disease is included in advance in, for example, rare disease information in a medical DB of a medical DB storage unit 33. The rare disease information may also include the number of clinical cases reported for the specific disease. The number of clinical cases reported for the specific disease may be stored in the memory 11 in advance. Further, when the number of clinical cases reported for the specific disease is equal to or less than a threshold value, the specific disease may be referred to as a rare disease. In other words, when the number of clinical cases reported for a specific disease is equal to or less than the threshold value, the foregoing description may be read while replacing the foregoing case with a case where the specific disease is a rare disease. Further, when the specific disease is a rare disease, the foregoing description may be read while replacing the foregoing case with a case where rare disease information is described for the specific disease.

Further, the display control function 15e causes the display 13 to display information indicating the specified hospital or doctor.

The other configurations are the same as in the first embodiment.

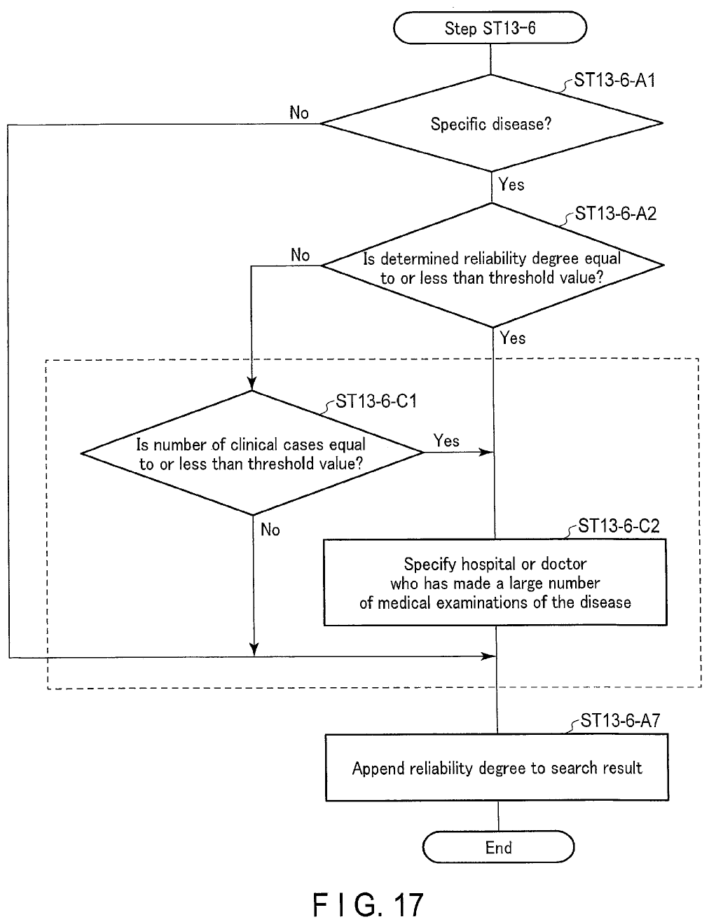

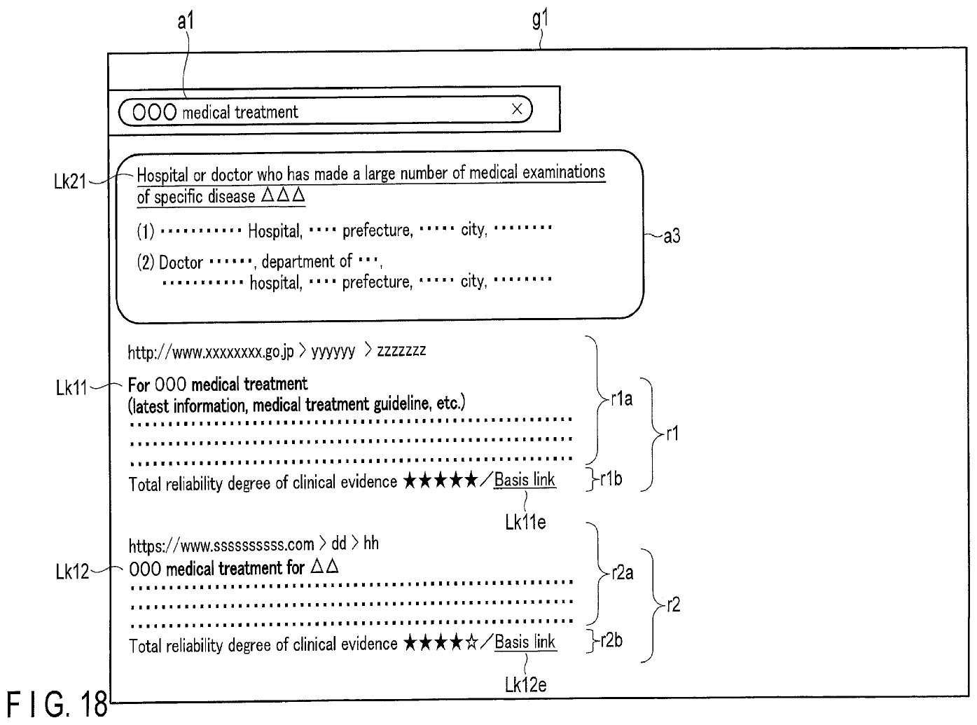

Next, an operation of the search information processing apparatus configured as described above will be described with reference to a flowchart of FIG. 17 and a schematic diagram of FIG. 18.

Now, it is assumed that steps ST1 to ST12 are executed in the same manner as described above, and descriptions corresponding to disease information set in step ST5 are extracted from each of search results of a search site.

After the step ST12, step ST13 for appending a reliability degree is executed as shown in FIG. 9. Steps ST13-1 to ST13-5 and ST13-7 of step ST13 are executed in the same manner as described above. On the other hand, step ST13-6 is executed, for example, by steps ST13-6-A1 to ST13-6-A2, ST13-6-C1 to ST13-6-C2, and ST13-6-A7 as shown in FIG. 17. In FIG. 17, a portion surrounded by a broken line is a portion that is significantly different from FIG. 14.

The processing circuitry 15 determines whether the disease information set in step ST5 indicates a specific disease (step ST13-6-A1). If the determination is negative, the processing circuitry 15 proceeds to step ST13-6-A7, and if the disease information indicates a specific disease, the processing circuitry 15 proceeds the next step ST13-6-A2.

The processing circuitry 15 determines whether the reliability degree determined in step ST13-5 is equal to or less than a threshold value (step ST13-6-A2). If the reliability degree is equal to or less than the threshold value, the processing circuitry 15 proceeds to step ST13-6-C2, and if the determination is negative, the processing circuitry 15 proceeds to the next step ST13-6-C1.

The processing circuitry 15 determines whether the number of clinical cases reported for the specific disease is equal to or less than a threshold value (step ST13-6-C1). If the determination is negative, the processing circuitry 15 proceeds to step ST13-6-A7, and if the number of clinical cases is equal to or less than the threshold value, the processing circuitry 15 proceeds to the next step ST13-6-C2.

The processing circuitry 15 specifies information indicating a hospital or doctor who has made a relatively large number of medical examinations of the specific disease (step ST13-6-C2). For example, the processing circuitry 15 reads out the search result of the medical DB stored in the memory 11 in step ST8 based on the disease name of the specific disease, and specifies information indicating the hospital or doctor from the rare disease information included in the read-out search result.

Thereafter, the processing circuitry 15 appends the reliability degree to the search result (step ST13 A7). As a result, the step ST13 ends.

Hereinafter, steps ST14 to ST18 are executed in the same manner as described above. However, in step ST17, the processing circuitry 15 causes the display 13 to display information indicating the specified doctor or hospital. For example, as shown in FIG. 18, the information indicating a hospital or doctor who has made a relatively large number of medical examinations of the specific disease is displayed in a display area a3 between the input area a1 of the search condition and the search result r1 at the top of the search. Further, for example, a link Lk21 relating to the information indicating the hospital or the doctor may be displayed in the intermediate display area a2.

As described above, according to the fourth embodiment, when disease information indicates a specific disease and the reliability degree of a search result is equal to or less than a threshold value or the number of clinical cases reported for the specific disease is equal to or less than a threshold value, information indicating a hospital or doctor who has made a relatively large number of medical examinations of the specific disease is specified. Further, the display is caused to display the information indicating the specified hospital or doctor.

In this way, even when the reliability degree of the search result for a specific disease is low or the number of clinical cases is small, in addition to the effect of the first embodiment, a doctor or a hospital from which the patient can receive a second opinion can be presented by the configuration of displaying information indicating a hospital or a doctor who has made a relatively large number of medical examinations of the specific disease. As a result, it is possible to support the patient in obtaining a second opinion.

According to at least one embodiment described above, when a disease is searched, it is possible to support appropriate selection of search results.

The term “processor” used in the above description means, for example, CPU (central processing unit), GPU (Graphics Processing Unit), or a circuit such as an application specific integrated circuit (ASIC), a programmable logic device (for example, simple programmable logic device (SPLD), a complex programmable logic device (CPLD), or a field programmable gate array (FPGA). The processor implements functions by reading and executing programs stored in a memory. Instead of storing the programs in the memory, the programs may be directly installed in the circuit of the processor. In this case, the processor implements the functions by reading and executing the program installed in the circuit. Note that each processor of the present embodiment is not limited to a case where each processor is configured as a single circuit, but a plurality of independent circuits may be combined to form a processor and implement the functions thereof. Further, a plurality of components in FIG. 1, 2 or 4 may be integrated into a processor to implement the function thereof.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A search information processing device comprising processing circuitry configured to:
set disease information of a patient;
extract descriptions corresponding to the disease information from each of search results of a search site on the Internet; and
append a reliability degree to each of the search results based on the extracted descriptions and evidence information for the disease information.

2. The search information processing device according to claim 1, wherein the processing circuitry is further configured to sort each of the search results to which the reliability degree is appended, according to the appended reliability degree and cause a display to display each of the sorted search results.

3. The search information processing device according to claim 2, wherein the processing circuitry is configured to control the display so as not to display a search result having an appended reliability degree equal to or less than a threshold value among the sorted search results.

4. The search information processing device according to claim 2, wherein the processing circuitry is configured to:
append a validity level of each of the extracted descriptions to the extracted description based on the evidence information; and
when any of the sorted search results is selected, cause the display to display descriptions included in the selected search result, and also cause the display to display the validity level appended to the extracted descriptions out of the descriptions.

5. The search information processing device according to claim 4, wherein the processing circuitry is configured to cause the display to display at least one of an explanation of the evidence information and a link destination of the evidence information.

6. The search information processing device according to claim 4, wherein each of the reliability degree and the validity level is an evidence level in a clinical research.

7. The search information processing device according to claim 1, wherein the processing circuitry is further configured to perform on/off-control on the extraction according to a user's operation.

8. The search information processing device according to claim 1, wherein the processing circuitry is configured to set the disease information based on an electronic medical record of the patient.

9. The search information processing device according to claim 1, wherein the processing circuitry is configured to: transmit a patient ID indicating the patient or a family ID indicating a family of the patient, and a terminal ID indicating a terminal of the patient or the family to a medical facility that the patient has visited or has been hospitalized in; receive the disease information as a response to the transmission; and set the received disease information.

10. The search information processing device according to claim 1, wherein the processing circuitry is configured to:
adjust a reliability degree before the reliability degree is appended to each of the search results, based on a number of related announced papers for a specific disease, a rank of a published journal of evidence information for the disease information, and a number of clinical cases reported for the specific disease when the disease information indicates the specific disease, and the reliability degree before the reliability degree is appended to the search result is equal to or less than a threshold value; and
append the adjusted reliability degree to the search result.

11. The search information processing device according to claim 2, wherein the processing circuitry is configured to:
specify both of a positive paper for a search result and a negative paper for the search result among related announced papers for a specific disease when the disease information indicates the specific disease and a reliability degree for the search result is equal to or less than a threshold value; and
cause a display to display information indicating both the specified positive and negative papers.

12. The search information processing device according to claim 2, wherein the processing circuitry is configured to:
specify information indicating a hospital or doctor who has made a relatively large number of medical examinations of a specific disease when the disease information indicates the specific disease and a reliability degree for the search result is equal to or less than a threshold value or when a number of clinical cases reported for the specific disease is equal to or less than a threshold value; and
cause the display to display information indicating the specified hospital or doctor.
